SEQUENCE LISTING (145 sequences)

SEQ ID NO: 1 - protein, 15 aa, Artificial sequence
  Description of Artificial Seguence Synthetic peptide of predeter mined sequence for testing of activity in MHC Class II assay
AAAYAAAAAAKAAAA

SEQ ID NO: 2 - protein, 15 aa, Artificial sequence
  Description of Artificial Sequence Synthetic peptide of predeter mined sequence for testing of activity in MHC Class II assay
AEKYAAAAAAKAAAA

SEQ ID NO: 3 - protein, 15 aa, Artificial Sequence
  Description of Artificial Sequence Synthetic peptide of predeter mined sequence for testing of activity in MHC Class II assays
AKEYAAAAAAKAAAA

SEQ ID NO: 4 - protein, 15 aa, Artificial Sequence
  Description of Artificial Sequence Synthetic peptide of predeter mined seguence for testing of activity in MHC class II assays
AKKYAAAAAAKAAAA

SEQ ID NO: 5 - protein, 15 aa, Artificial Sequence
  Description of Artificial Sequence Synthetic peptide of predeter mined sequence for testing of activity in MHC Class II assays
AEAYAAAAAAKAAAA

SEQ ID NO: 6 - protein, 15 aa, Artificial Sequence
  Description of Artificial Sequence Synthetic peptide of predeter mined seguence for testing of activity in MHC class II assays
KEAYAAAAAAKAAAA

SEQ ID NO: 7 - protein, 15 aa, Artificial Sequence
  Description of Artificial Sequence Synthetic peptide of predeter mined seguence for testing of activity in MHC class II assays
AEEYAAAAAAKAAAA

SEQ ID NO: 8 - protein, 15 aa, Artificial Sequence
  Description of Artificial Sequence Synthetic peptide of predeter mined seguence for testing of activity in MHC class II assays
AAEYAAAAAAKAAAA

SEQ ID NO: 9 - protein, 15 aa, Artificial Sequence
  Description of Artificial Sequence Synthetic peptide of predeter mined seguence for testing of activity in MHC class II assays.
EKAYAAAAAAKAAAA

SEQ ID NO: 10 - protein, 15 aa, Artificial Sequence
  Description of Artificial Sequence Synthetic peptide of predeter mined seguence for testing of activity in MHC class II assays
AAKYEAAAAAKAAAA

SEQ ID NO: 11 - protein, 15 aa, Artificial Sequence
  Description of Artificial Sequence Synthetic peptide of predeter mined seguence for testing of activity in MHC class II assays
AAKYAEAAAAKAAAA

SEQ ID NO: 12 - protein, 15 aa, Artificial Sequence
  Description of Artificial Sequence Synthetic peptide of predeter mined seguence for testing of activity in MHC class II assays
EAAYAAAAAAKAAAA

SEQ ID NO: 13 - protein, 15 aa, Artificial Sequence
  Synthetic peptide of predetermined seguence for testing of activity in MHC class II assays
EKKYAAAAAAKAAAA

SEQ ID NO: 14 - protein, 15 aa, Artificial Sequence
  Description of Artificial Sequence Synthetic peptide of predeter mined seguence for testing of activity in MHC class II assays
EAKYAAAAAAKAAAA

SEQ ID NO: 15 - protein, 15 aa, Artificial Sequence
  Description of Artificial Sequence Synthetic peptide of predeter mined seguence for testing of activity in MHC class II assays
AEKYAAAAAAAAAAA

SEQ ID NO: 16 - protein, 15 aa, Artificial Sequence
  Description of Artificial Sequence Synthetic peptide of predeter mined seguence for testing of activity in MHC class II assays
AKEYAAAAAAKAAAA

SEQ ID NO: 17 - protein, 15 aa, Artificial Sequence
  Description of Artificial Sequence Synthetic peptide of predeter mined sequence for testing of activity in MHC class II assays
AKKYEAAAAAAAAAA

SEQ ID NO: 18 - protein, 15 aa, Artificial Sequence
  Description of Artificial Sequence Synthetic peptide of predeter mined sequence for testing of activity in MHC class II assays
AKKYAEAAAAAAAAA

SEQ ID NO: 19 - protein, 15 aa, Artificial Sequence
  Description of Artificial Sequence Synthetic peptide of predeter mined sequence for testing of activity in MHC class II assays
AEAYKAAAAAAAAAA

SEQ ID NO: 20 - protein, 15 aa, Artificial Sequence
  Description of Artificial Sequence Synthetic peptide of predeter mined sequence for testing of activity in MHC class II assays
KEAYAAAAAAAAAAA

SEQ ID NO: 21 - protein, 15 aa, Artificial Sequence
  Description of Artificial Sequence Synthetic peptide of predeter mined sequence for testing of activity in MHC class II assays
AEEYKAAAAAAAAAA

SEQ ID NO: 22 - protein, 15 aa, Artificial Sequence
  Description of Artificial Sequence Synthetic peptide of predeter mined sequence for testing of activity in MHC class II assays
AAEYKAAAAAAAAAA

SEQ ID NO: 23 - protein, 15 aa, Artificial Sequence
  Description of Artificial Sequence Synthetic peptide of predeter mined sequence for testing of activity in MHC class II assays
EKAYAAAAAAAAAAA

SEQ ID NO: 24 - protein, 15 aa, Artificial Sequence
  Description of Artificial Sequence Synthetic peptide of predeter mined sequence for testing of activity in MHC class II assays
AAKYEAAAAAAAAAA

SEQ ID NO: 25 - protein, 15 aa, Artificial Sequence
  Description of Artificial Sequence Synthetic peptide of predeter mined sequence for testing of activity in MHC class II assays
AAKYAEAAAAAAAAA

SEQ ID NO: 26 - protein, 15 aa, Artificial Sequence
  Description of Artificial Sequence Synthetic peptide of predeter mined sequence for testing of activity in MHC class II assays
EKKYAAAAAAAAAAA

SEQ ID NO: 27 - protein, 15 aa, Artificial Sequence
  Description of Artificial Sequence Synthetic peptide of predeter mined seguence for testing of activity in MHC class II assays
EAKYAAAAAAAAAAA

SEQ ID NO: 28 - protein, 15 aa, Artificial Sequence
  Description of Artificial Sequence Synthetic peptide of predeter mined sequence for testing of activity in MHC class II assays
AEYAKAAAAAAAAAA

SEQ ID NO: 29 - protein, 15 aa, Artificial Sequence
  Description of Artificial Sequence Synthetic peptide of predeter mined sequence for testing of activity in MHC class II assays
AEKAYAAAAAAAAAA

SEQ ID NO: 30 - protein, 15 aa, Artificial Sequence
  Description of Artificial Sequence Synthetic peptide of predeter mined sequence for testing of activity in MHC class II assays
EKYAAAAAAAAAAAA

SEQ ID NO: 31 - protein, 15 aa, Artificial Sequence
  Description of Artificial Sequence Synthetic peptide of predeter mined sequence for testing of activity in MHC class II assays
AYKAEAAAAAAAAAA

SEQ ID NO: 32 - protein, 15 aa, Artificial Sequence
  Description of Artificial Sequence Synthetic peptide of predeter mined seguence for testing of activity in MHC class II assays
AKYAEAAAAAAAAAA

SEQ ID NO: 33 - protein, 4 aa, Artificial Sequence
  Description of Artificial Sequence Synthetic peptide of predeter mined sequence for testing of activity in MHC class II assays
EKVA

SEQ ID NO: 34 - protein, 4 aa, Artificial Sequence
  Description of Artificial Sequence Synthetic peptide of predeter mined sequence for testing of activity in MHC class II assays
EKFA

SEQ ID NO: 35 - protein, 5 aa, Artificial Sequence
  Description of Artificial Sequence Synthetic peptide of predeter mined sequence for testing of activity in MHC class II assays
AEKYA

SEQ ID NO: 36 - protein, 5 aa, Artificial Sequence
  Description of Artificial Sequence Synthetic peptide of predeter mined sequence for testing of activity in MHC class II assays
AEKVA

SEQ ID NO: 37 - protein, 5 aa, Artificial Sequence
  Description of Artificial Sequence Synthetic peptide of predeter mined sequence for testing of activity in MHC class II assays
AEKFA

SEQ ID NO: 38 - protein, 4 aa, Artificial Sequence
  Description of Artificial Sequence Synthetic peptide of predeter mined sequence for testing of activity in MHC class II assays
KEYA

SEQ ID NO: 39 - protein, 4 aa, Artificial Sequence
  Description of Artificial Sequence Synthetic peptide of predeter mined sequence for testing of activity in MHC class II assays
KYAE

SEQ ID NO: 40 - protein, 4 aa, Artificial Sequence
  Description of Artificial Sequence Synthetic peptide of predeter mined sequence for testing of activity in MHC class II assays
KEVA

SEQ ID NO: 41 - protein, 4 aa, Artificial Sequence
  Description of Artificial Sequence Synthetic peptide of predeter mined sequence for testing of activity in MHC class II assays
KVAE

SEQ ID NO: 42 - protein, 4 aa, Artificial Sequence
  Description of Artificial Sequence Synthetic peptide of predeter mined sequence for testing of activity in MHC class II assays
KEFA

SEQ ID NO: 43 - protein, 4 aa, Artificial Sequence
  Description of Artificial Sequence Synthetic peptide of predeter mined sequence for testing of activity in MHC class II assays
KFAE

SEQ ID NO: 44 - protein, 4 aa, Artificial Sequence
  Description of Artificial Sequence Synthetic peptide of predeter mined sequence for testing of activity in MHC class II assays
KYAA

SEQ ID NO: 45 - protein, 4 aa, Artificial Sequence
  Description of Artificial Sequence Synthetic peptide of predeter mined sequence for testing of activity in MHC class II assays
KKYA

SEQ ID NO: 46 - protein, 4 aa, Artificial Sequence
  Description of Artificial Sequence Synthetic peptide of predeter mined sequence for testing of activity in MHC class II assays
KVAA

SEQ ID NO: 47 - protein, 4 aa, Artificial Sequence
  Description of Artificial Sequence Synthetic peptide of predeter mined sequence for testing of activity in MHC class II assays
KKVA

SEQ ID NO: 48 - protein, 4 aa, Artificial Sequence
  Description of Artificial Sequence Synthetic peptide of predeter mined sequence for testing of activity in MHC class II assays
KFAA

SEQ ID NO: 49 - protein, 4 aa, Artificial Sequence
  Description of Artificial Sequence Synthetic peptide of predeter mined sequence for testing of activity in MHC class II assays
KKFA

SEQ ID NO: 50 - protein, 5 aa, Artificial Sequence
  Description of Artificial Sequence Synthetic peptide of predeter mined sequence for testing of activity in MHC class II assays
AKYAE

SEQ ID NO: 51 - protein, 5 aa, Artificial Sequence
  Description of Artificial Sequence Synthetic peptide of predeter mined sequence for testing of activity in MHC class II assays
EAKYA

SEQ ID NO: 52 - protein, 5 aa, Artificial Sequence
  Description of Artificial Sequence Synthetic peptide of predeter mined sequence for testing of activity in MHC class II assays
AKVAE

SEQ ID NO: 53 - protein, 5 aa, Artificial Sequence
  Description of Artificial Sequence Synthetic peptide of predeter mined sequence for testing of activity in MHC class II assays
EAKVA

SEQ ID NO: 54 - protein, 5 aa, Artificial Sequence
  Description of Artificial Sequence Synthetic peptide of predeter mined sequence for testing of activity in MHC class II assays
AKFAE

SEQ ID NO: 55 - protein, 5 aa, Artificial Sequence
  Description of Artificial Sequence Synthetic peptide of predeter mined sequence for testing of activity in MHC class II assays
EAKFA

SEQ ID NO: 56 - protein, 23 aa, Artificial Sequence
  Synthetic peptide - MOG 43-56 - containing the nonameric core sequence with preferred binding mode to the HLA-DR/DQ molecule
GMEVGWYRPPPSRVVHLYRNGKD

SEQ ID NO: 57 - protein, 48 aa, Artificial Sequence
  Synthetic peptide - MOG 67-114 - containing the nonameric core sequence with preferred binding mode to the HLA-DR/DQ molecule
GRTELLKDAIGEGKVTLRIRNVRPSDEGGPTSPPRDHSYQEEAAMELK

SEQ ID NO: 58 - protein, 25 aa, Artificial Sequence
  Synthetic peptide - MOG 205-215 - containing the nonameric core sequence with preferred binding mode to the HLA-DR/DQ molecule
PRVIGPRHPIRALVGDEVELPSRIS

SEQ ID NO: 59 - protein, 11 aa, Artificial Sequence
  Synthetic peptide - MOG 205-215 - containing the nonameric core sequence with preferred binding mode to the HLA-DR/DQ molecule
RLAGQPLEELR

SEQ ID NO: 60 - protein, 28 aa, Artificial Sequence
  Synthetic peptide - MBP 84-111 - containing the nonameric core sequence with preferred binding mode to the HLA-DR/DQ molecule
NPVVHPPKNIVTPRTPPPSQGKGRGLSL

SEQ ID NO: 61 - protein, 28 aa, Artificial Sequence
  Synthetic peptide - MEP 141-168 - containing the nonameric core sequence with preferred binding mode to the HLA-DR/DQ molecule
PKGVDAQGTLSKIPKLGGRDSRSGSPMA

SEQ ID NO: 62 - protein, 31 aa, Artificial Sequence
  Synthetic peptide - MBP 12-42 - containing the nonameric core sequence with preferred binding mode to the HLA-DR/DQ molecule
SKYLATASTMDHARHGPLPRHRDTGILDSIG

SEQ ID NO: 63 - protein, 32 aa, Artificial Sequence
  Synthetic peptide - OSP 42-73 - containing the nonameric core sequence with preferred binding mode to the HLA-DR/DQ molecule
KLDELGSKGLWADSVMATGLYHSKPLVDILIL

SEQ ID NO: 64 - protein, 12 aa, Artificial Sequence
  Synthetic peptide - OSP 98-109 - containing the nonameric core sequence with preferred binding mode to the HLA-DR/DQ molecule
LLTVLPSIRMGQ

SEQ ID NO: 65 - protein, 20 aa, Artificial Sequence
  Synthetic peptide - OSP 20-33 - containing the nonameric core sequence with preferred binding mode to the HLA-DR/DQ molecule
NRPYYTAGSSSPTHAKSAHV

SEQ ID NO: 66 - protein, 15 aa, Artificial Sequence
  Synthetic peptide - OSP 192-206 - containing the nonameric core sequence with preferred binding mode to the HLA-DR/DQ molecule
TAGSSSPTHAKSAHV

SEQ ID NO: 67 - protein, 14 aa, Artificial Sequence
  Synthetic peptide - OSP 20-33 - containing the nonameric core sequence with preferred binding mode to the HLA-DR/DQ molecule
VIVTTSTNDWVVTS

SEQ ID NO: 68 - protein, 17 aa, Artificial Sequence
  Synthetic peptide - OSP 129-145 - containing the nonameric core sequence with preferred binding mode to the HLA-DR/DQ molecule
LALSALVATIWPPVSAH

SEQ ID NO: 69 - protein, 19 aa, Artificial Sequence
  Synthetic peptide - MOBP 15-33 - containing the nonameric core sequence with preferred binding mode to the HLA-DR/DQ molecule
QKYSEHPSIHSSPPPTPLN

SEQ ID NO: 70 - protein, 36 aa, Artificial Sequence
  Synthetic peptide - MOBP 55-90 - containing the nonameric core sequence with preferred binding mode to the HLA-DR/DQ molecule.
KEEDWISSASQKTRTSRRAKSPQRPKQQPAAPPAVV

SEQ ID NO: 71 - protein, 17 aa, Artificial Sequence
  Synthetic peptide - MOBP 156-172 - containing the nonameric core sequence with preferred binding mode to the HLA-DR/DQ molecule
KQQPRSSPLRGPGASRG

SEQ ID NO: 72 - protein, 48 aa, Artificial Sequence
  Synthetic peptide - PLP 103-150 - containing the nonameric core sequence with preferred binding mode to the HLA-DR/DQ molecule
YKTTISGKGLSATVTGGQKGRGSRGQHQAHSLERVSHSLGKWLGHPDK

SEQ ID NO: 73 - protein, 27 aa, Artificial Sequence
  Synthetic peptide - PLP 177-203 - containing the nonameric core sequence with preferred binding mode to the HLA-DR/DQ molecule
PNTWTTSQSIAPPSKTSASIGSLSADA

SEQ ID NO: 74 - protein, 23 aa, Artificial Sequence
  Synthetic peptide - PLP 218-240 - containing the nonameric core sequence with preferred binding mode to the HLA-DR/DQ molecule
VSGSNLLSISKTAEPQMTPHLPI

SEQ ID NO: 75 - protein, 15 aa, Artificial Sequence
  Synthetic peptide - 38-52 -containing the nonameric core sequence with preferred binding mode to the HLA-DR/DQ molecule
ALTGTEKLIETYPSK

SEQ ID NO: 76 - protein, 12 aa, Artificial Sequence
  Synthetic peptide - PLP 264-276 - containing the nonameric core sequence with preferred binding mode to the HLA-DR/DQ molecule
PAVLKMGRGTKP

SEQ ID NO: 77 - protein, 22 aa, Artificial Sequence
  Synthetic peptide - MOG 37-58 - containing the nonameric core sequence which fits into the MS-relevant HLA-DR/DQ molecule and are flanked by 2-5 amino acids at their N- and C-termini
VGWYRPPPSRVVHLYRNGKDQD

SEQ ID NO: 78 - protein, 31 aa, Artificial Sequence
  Synthetic peptide - MOG 65-95 - containing the nonameric core sequence which fits into the MS-relevant HLA-DR/DQ molecule and are flanked by 2-5 amino acids at their N- and C-termini
YRGRTELLKDAIGEGKVTLRIRNVRPSDEGG

SEQ ID NO: 79 - protein, 26 aa, Artificial Sequence
  Synthetic peptide - MOG 7-32 - containing the nonameric core sequence which fits into the MS-relevant HLA-DR/DQ molecule and are flanked by 2-5 amino acids at their N- and C-termini
GPRHPIRALVGDEVELRCRISPGKNA

SEQ ID NO: 80 - protein, 17 aa, Artificial Sequence
  Synthetic peptide - MOG 202-218 - containing the nonameric core sequence which fits into the MS-relevant HLA-DR/DQ molecule and are flanked by 2-5 amino acids at their N and C-termini
LHRRLAGQPLEELRNPP

SEQ ID NO: 81 - protein, 22 aa, Artificial Sequence
  Synthetic peptide - MBP 82-103 - containing the nonameric core sequence which fits into the MS-relevant HLA-DR/DQ molecule and are flanked by 2-5 amino acids at their N- and C-termini
DENPVVHPPKNIVTPRTPPPSQ

SEQ ID NO: 82 - protein, 21 aa, Artificial Sequence
  Synthetic peptide - MBP 136-156 - containing the nonameric core sequence which fits into the MS-relevant HLA-DR/DQ molecule and are flanked by 2-5 amino acids at their N and C-termini
SAHKGPKGVDAQGTLSKIPKL

SEQ ID NO: 83 - protein, 23 aa, Artificial Sequence
  Synthetic peptide - MBP 148-170 - containing the nonameric core sequence which fits into the MS-relevant HLA-DR/DQ molecule and are flanked by 2-5 amino acids at their N and C-termini
GTLSKIPKLGGRDSRSGSPMARR

SEQ ID NO: 84 - protein, 23 aa, Artificial Sequence
  Synthetic peptide - MBP 7-29 - containing the nonameric core sequence which fits into the MS-relevant HLA-DR/DQ molecule and are flanked by 2-5 amino acids at their N- and C-termini
SQRHGSKYLATASTMDHARHGPL

SEQ ID NO: 85 - protein, 21 aa, Artificial Sequence
  Synthetic peptide - MBP 25-45 - containing the nonameric core sequence which fits into the MS-relevant HLA-DR/DQ molecule and are flanked by 2-5 amino acids at their N- and C-termini
RHGPLPRHRDTGILDSIGRPP

SEQ ID NO: 86 - protein, 30 aa, Artificial Sequence
  Synthetic peptide - OSP 38-64 - containing the nonameric core sequence which fits into the MS-relevant HLA-DR/DQ molecule and are flanked by 2-5 amino acids at their N- and C-termini
SKGLWADCVMATGLYHCKPLVDILILPGYV

SEQ ID NO: 87 - protein, 27 aa, Artificial Sequence
  Synthetic peptide - OSP 48-77 - containing the nonameric core sequence which fits into the MS-relevant HLA-DR/DQ molecule and are flanked by 2-5 amino acids at their N- and C-termini
PTCRKLDELGSKGLWADCVMATGLYHC

SEQ ID NO: 88 - protein, 19 aa, Artificial Sequence
  Synthetic peptide - OSP 94-112 - containing the nonameric core sequence which fits into the MS-relevant HLA-DR/DQ molecule and are flanked by 2-5 amino acids at their N- and C-termini
AILLLLTVLPCIRMGQEPG

SEQ ID NO: 89 - protein, 22 aa, Artificial Sequence
  Synthetic peptide - OSP 17-38 - containing the nonameric core sequence which fits into the MS-relevant HLA-DR/DQ molecule and are flanked by 2-5 amino acids at their N- and C-termini
WIGVIVTTSTNDWVVTCGYTIP

SEQ ID NO: 90 - protein, 27 aa, Artificial Sequence
  Synthetic peptide - OSP 124-150 - containing the nonameric core sequence which fits into the MS-relevant HLA-DR/DQ molecule and are flanked by 2-5 amino acids at their N- and C-termini
VLLILLALCALVATIWPPVCAHRETTI

SEQ ID NO: 91 - protein, 26 aa, Artificial Sequence
  Synthetic peptide - MOBP 13-38 - containing the nonameric core sequence which fits into the MS-relevant HLA-DR/DQ molecule and are flanked by 2-5 amino acids at their N- and C-termini
KNQKYSEHPSIHCCPPPTPLNSKKEI

SEQ ID NO: 92 - protein, 27 aa, Artificial Sequence
  Synthetic peptide - MOBP 54-80 - containing the nonameric core sequence which fits into the MS-relevant HLA-DR/DQ molecule and are flanked by 2-5 amino acids at their N- and C-termini
QKEEDWTCCACQKTRTSRRAKSPQRPK

SEQ ID NO: 93 - protein, 18 aa, Artificial Sequence
  Synthetic peptide - MOBP 72-89 - containing the nonameric core sequence which fits into the MS-relevant HLA-DR/DQ molecule and are flanked by 2-5 amino acids at their N- and C-termini
RAKSPQRPKQQPAAPPAV

SEQ ID NO: 94 - protein, 19 aa, Artificial Sequence
  Synthetic peptide - MOBP 156-174 - containing the nonameric core sequence which fits into the MS-relevant HLA-DR/DQ molecule and are flanked by 2-5 amino acids at their N-and C-termini
KQQPRSSPLRGPGASRGGS

SEQ ID NO: 95 - protein, 21 aa, Artificial Sequence
  Synthetic peptide - PLP 102-122 - containing the nonameric core sequence which fits into the MS-relevant HLA-DR/DQ molecule and are flanked by 2-5 amino acids at their N-and C-termini
DYKTTICGKGLSATVTGGQKG

SEQ ID NO: 96 - protein, 31 aa, Artificial Sequence
  Synthetic peptide - PLP 120-150 - containing the nonameric core sequence which fits into the MS-relevant HLA-DR/DQ molecule and are flanked by 2-5 amino acids at their N-and C-termini
QKGRGSRGQHQAHSLERVCHCLGKWLGHPDK

SEQ ID NO: 97 - protein, 28 aa, Artificial Sequence
  Synthetic peptide - PLP 173-200 - containing the nonameric core sequence which fits into the MS-relevant HLA-DR/DQ molecule and are flanked by 2-5 amino acids at their N-and C-termini
VYIYPNTWTTCQSIAPPSKTSASIGSLC

SEQ ID NO: 98 - protein, 19 aa, Artificial Sequence
  Synthetic peptide - PLP 190-208  - containing the nonameric core sequence which fits into the MS-relevant HLA-DR/DQ molecule and are flanked by 2-5 amino acids at their N-and C-termini
SKTSASIGSLCADARMYGV

SEQ ID NO: 99 - protein, 22 aa, Artificial Sequence
  Synthetic peptide - PLP 185-206 - containing the nonameric core sequence which fits into the MS-relevant HLA-DR/DQ molecule and are flanked by 2-5 amino acids at their N-and C-termini
SIAPPSKTSASIGSLCADARMY

SEQ ID NO: 100 - protein, 31 aa, Artificial Sequence
  Synthetic peptide - PLP 213-243 - containing the nonameric core sequence which fits into the MS-relevant HLA-DR/DQ molecule and are flanked by 2-5 amino acids at their N-and C-termini
APPGKVCGSNLLSICKTAEPQMTPHLPIAAP

SEQ ID NO: 101 - protein, 16 aa, Artificial Sequence
  Synthetic peptide - PLP 35-57 - containing the nonameric core sequence which fits into the MS-relevant HLA-DR/DQ molecule and are flanked by 2-5 amino acids at their N-and C-termini
TYNPAVLKLMGRGTKP

SEQ ID NO: 102 - protein, 23 aa, Artificial Sequence
  Synthetic peptide - PLP 261-276 - containing the nonameric core sequence which fits into the MS-relevant HLA-DR/DQ molecule and are flanked by 2-5 amino acids at their N-and C-termini
GHEALTGTEKLIETYPSKNYQDY

SEQ ID NO: 103 - protein, 22 aa, Artificial Sequence
  The peptide of SEQ ID NO 77 in which the residues at positions 7 and 13 were replaced by  A
VGWYRPAPSRVVALYRNGKDQD

SEQ ID NO: 104 - protein, 19 aa, Artificial Sequence
  The peptide of SEQ ID NO 77 in which the residue at position 10 was replaced by  A  and the three residues at the C-terminus are lacking
VGWYRPPPSAVVHLYRNGK

SEQ ID NO: 105 - protein, 31 aa, Artificial Sequence
  The peptide of SEQ ID NO 78 in which the residues at positions 9, 17, 22 were replaced by  A
YRGRTELLADAIGEGKATLRIANVRPSDEGG

SEQ ID NO: 106 - protein, 20 aa, Artificial Sequence
  The peptide of SEQ ID NO 78 in which the residues at positions 6 and 11 were replaced by  A , 12 residues at the N-terminus are lacking, and having additional  P  at the C-terminus
GEGKVALRIRAVRPSDEGGP

SEQ ID NO: 107 - protein, 26 aa, Artificial Sequence
  The peptide of SEQ ID NO 79 in which the residues at positions 7 and 17 were replaced by  A
GPRHPIAALVGDEVELACRISPGKNA

SEQ ID NO: 108 - protein, 16 aa, Artificial Sequence
  The peptide of SEQ ID NO 80 in which the residue at position 9 was replaced by  A
LHRRLAGQALEELRNP

SEQ ID NO: 109 - protein, 22 aa, Artificial Sequence
  The peptide of SEQ ID NO 81 in which the residue at position 10 was replaced by A
DENPVVHPPANIVTPRTPPPSQ

SEQ ID NO: 110 - protein, 21 aa, Artificial Sequence
  The peptide of SEQ ID NO 81 in which the residues at positions 6 and 12 were replaced by  A , and one residue at the N-terminus is lacking
ENPVVAPPKNIATPRTPPPSQ

SEQ ID NO: 111 - protein, 21 aa, Artificial Sequence
  The peptide of SEQ ID NO 82 in which the residue at position 10 was replaced by  A .
SAHKGPKGVAAQGTLSKIPKL

SEQ ID NO: 112 - protein, 23 aa, Artificial Sequence
  The peptide of SEQ ID NO 83 in which the residues at positions 7 and 13 were replaced by  A
GTLSKIAKLGGRASRSGSPMARR

SEQ ID NO: 113 - protein, 23 aa, Artificial Sequence
  The peptide of SEQ ID NO 82 in which the residues at positions 7 and 14 were replaced by  A , and having additional 2 residues at the C-terminus
SAHKGPAGVDAQGALSKIPKLGG

SEQ ID NO: 114 - protein, 23 aa, Artificial Sequence
  The peptide of SEQ ID NO 84 in which the residues at positions 7 and 17 were replaced by  A .
SQRHGSAYLATASTMDAARHGPL

SEQ ID NO: 115 - protein, 21 aa, Artificial Sequence
  The peptide of SEQ ID NO 85 in which the residue at position 10 was replaced by  A
RHGPLPRHRATGILDSIGRPP

SEQ ID NO: 116 - protein, 19 aa, Artificial Sequence
  The peptide of SEQ ID NO 85 in which the residue at position 7 was replaced by  A , and 2 residues at the N-terminus are lacking.
GPLPRHADTGILDSIGRPP

SEQ ID NO: 117 - protein, 30 aa, Artificial Sequence
  The peptide of SEQ ID NO 86 in which the residues at positions 5, 10, 16 and 21 were replaced by  A
SKGLAADCVAATGLYACKPLADILILPGYV

SEQ ID NO: 118 - protein, 27 aa, Artificial Sequence
  The peptide of SEQ ID NO 87 in which the residues at positions 7, 15, 20 and 26 were replaced by  A .
PTCRKLAELGSKGLAADCVAATGLYAC

SEQ ID NO: 119 - protein, 26 aa, Artificial Sequence
  The peptide of SEQ ID NO 87 in which the residues at positions 6, 11 and 16 were replaced by  A  and one residue at the N-terminus is lacking
TCRKLAELGSAGLWAACVMATGLYHC

SEQ ID NO: 120 - protein, 19 aa, Artificial Sequence
  The peptide of SEQ ID NO 88 in which the residues at position 10 was replaced by  A
AILLLLTVLACIRMGQEPG

SEQ ID NO: 121 - protein, 20 aa, Artificial Sequence
  The peptide of SEQ ID NO 65 in which the residues at positions 10 and 16 were replaced by  A
NRPYYTAGSASPTHAASAHV

SEQ ID NO: 122 - protein, 15 aa, Artificial Sequence
  The peptide of SEQ ID NO 65 in which the residue at position 9 was replaced by  A  and 5 residues at the N-terminus are lacking.
TAGSSSPTAAKSAHV

SEQ ID NO: 123 - protein, 22 aa, Artificial Sequence
  The peptide of SEQ ID NO 89 in which the residue at position 10 was replaced by  A
WIGVIVTTSANDWVVTCGYTIP

SEQ ID NO: 124 - protein, 27 aa, Artificial Sequence
  The peptide of SEQ ID NO 90 in which the residues at positions 8 and 18 were replaced by  A
VLLILLAACALVATIWPAVCAHRETTI

SEQ ID NO: 125 - protein, 26 aa, Artificial Sequence
  The peptide of SEQ ID NO 91 in which the residues at positions 7 and 17 were replaced by  A
KNQKYSAHPSIHCCPPATPLNSKKEI

SEQ ID NO: 126 - protein, 26 aa, Artificial Sequence
  The peptide of SEQ ID NO 92 in which the residues at positions 6, 12 and 17 were replaced by  A  and one residue at the N-terminus is lacking
KEEDWACCACQATRTSARAKSPQRPK

SEQ ID NO: 127 - protein, 19 aa, Artificial Sequence
  The peptide of SEQ ID NO 94 in which the residue at position 10 was replaced by  A .
KQQPRSSPLAGPGASRGGS

SEQ ID NO: 128 - protein, 18 aa, Artificial Sequence
  The peptide of SEQ ID NO 93 in which the residue at position 9 was replaced by  A .
RAKSPQRPAQQPAAPPAV

SEQ ID NO: 129 - protein, 16 aa, Artificial Sequence
  The peptide of SEQ ID NO 95 in which the residue at position 9 was replaced by  A
DYKTTICGAGLSATVT

SEQ ID NO: 130 - protein, 21 aa, Artificial Sequence
  The peptide of SEQ ID NO 95 in which the residue at position 11 was replaced by  A
DYKTTICGKGASATVTGGQKG

SEQ ID NO: 131 - protein, 31 aa, Artificial Sequence
  The peptide of SEQ ID NO 96 in which the residues at positions 10, 15 and 25 were replaced by  A
QKGRGSRGQAQAHSAERVCHCLGKALGHPDK

SEQ ID NO: 132 - protein, 28 aa, Artificial Sequence
  The peptide of SEQ ID NO 97 in which the residues at positions 10 and 19 were replaced by  A
VYIYPNTWTACQSIAPPSATSASIGSLC

SEQ ID NO: 133 - protein, 19 aa, Artificial Sequence
  The peptide of SEQ ID NO 98 in which the residue at position 10 was replaced by  A
SKTSASIGSACADARMYGV

SEQ ID NO: 134 - protein, 24 aa, Artificial Sequence
  The peptide of SEQ ID NO 97 in which the residues at positions 6, 12 and 18 were replaced by  A , 2 residues at the N-terminus and 2 residues as the C-terminus are lacking.
IYPNTATTCQSAAPPSKASASIGS

SEQ ID NO: 135 - protein, 22 aa, Artificial Sequence
  The peptide of SEQ ID NO 99 in which the residues at positions 2, 8 and 13 were replaced by  A
SAAPPSKASASIASLCADARMY

SEQ ID NO: 136 - protein, 20 aa, Artificial Sequence
  The peptide of SEQ ID NO 97 in which the residues at positions 7 and 15 were replaced by  A , the  C  at position 1 was replaced by  S , having additional 2 residues at the C-terminus and 10 residues at the N-terminus are lacking.
SQSIAPASKTSASIASLCAD

SEQ ID NO: 137 - protein, 22 aa, Artificial Sequence
  The peptide of SEQ ID NO 45 in which the residue at position 10 was replaced by  A  and 9 residues at the C-terminus are lacking.
APPGKVCGSALLSICKTAEPQM

SEQ ID NO: 138 - protein, 19 aa, Artificial Sequence
  The peptide of SEQ ID NO 100 in which the residue at position 10 was replaced by  A  and 12 residues at the N-terminus are lacking
SICKTAEPQATPHLPIAAP

SEQ ID NO: 139 - protein, 23 aa, Artificial Sequence
  The peptide of SEQ ID NO 100 in which the residues at positions 6 and 11 were replaced by  A  and 8 residues at the C-terminus are lacking.
APPGKACGSNALSICKTAEPQMT

SEQ ID NO: 140 - protein, 16 aa, Artificial Sequence
  The peptide of SEQ ID NO 101 in which the residue at position 10 was replaced by  A
TYNPAVLKLAGRGTKP

SEQ ID NO: 141 - protein, 23 aa, Artificial Sequence
  The peptide of SEQ ID NO 102 in which the residue at position 10 was replaced by  A
GHEALTGTEALIETYPSKNYQDY

SEQ ID NO: 142 - protein, 5 aa, Artificial Sequence
  Description of Artificial Sequence Synthetic peptide of predeter mined sequence for testing of activity in MHC Class II assays
AKVAE

SEQ ID NO: 143 - protein, 5 aa, Artificial Sequence
  Description of Artificial Sequence Synthetic peptide of predeter mined sequence for testing of activity in MHC class II assays
EAKVA

SEQ ID NO: 144 - protein, 5 aa, Artificial Sequence
  Description of Artificial Sequence Synthetic peptide of predeter mined sequence for testing of activity in MHC class II assays
AKFAE

SEQ ID NO: 145 - protein, 5 aa, Artificial Sequence
  Description of Artificial Sequence Synthetic peptide of predeter mined sequence for testing of activity in MHC class II assays
EAKFA

(19) United States
(12) Patent Application Publication
(10) Pub. No.: US 2006/0040899 A1
(43) Pub. Date: Feb. 23, 2006

(54) Medicaments containing bisphosphonic acids and derivatives thereof for preventing and treating diseases and allergies

(76) Inventor: Hassan Jomaa, Giessen (DE)

Correspondence Address: HARNESS, DICKEY & PIERCE, P.L.C., P.O. BOX 8910, RESTON, VA, 20195, US

(21) Appl. No.: 11/121,044

(22) Filed: May 4, 2005

Related U.S. Application Data
(63) Continuation-in-part of application No. 09/719,946, filed on Dec. 15, 2000, now abandoned.

(51) Int. Cl. A61K 31/675; A61K 31/663

(52) U.S. Cl. 514/79; 514/102

(57) ABSTRACT

Provided are compounds and methods for the prevention and treatment of autoimmune disorders and of allergies using such compositions in which autoantigens or allergens previously used for treating autoimmune disorders and allergies are used in combination with bisphosphonates or the derivatives thereof. Bisphosphonic acids and derivatives thereof generally corresponding to Formula I illustrated below are useful in the production of pharmaceutical formulations that may be used for the prevention and treatment of various autoimmune diseases or allergies. The bisphosphonic acids and the derivatives thereof which are used are those represented by the Formula I: 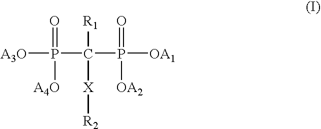 in which the variables A1, A2, A3, A4, R1, R2 and X are selected from a range of substituents as outlined in the specification.

This application is a continuation-in-part of, and hereby claims priority under 35 U.S.C. § 120 from U.S. application Ser. No. 09/719,946, the entire contents of which are hereby incorporated herein by reference.

### FIELD OF THE INVENTION

This invention relates to pharmaceutical preparations for the prevention and treatment of autoimmune disorders and of allergies.

It is known that autoimmune disorders, such as multiple sclerosis, rheumatoid arthritis, diabetes mellitus, myasthenia gravis, uveitis, and allergies, in particular food allergies, nickel allergy and pollen allergies, are attributable to an inappropriate reaction by the body's immune system.

It is furthermore known that, due to these inappropriate reactions of the immune system, endogenous substances (autoantigens) are perceived as foreign substances and a defense reaction develops against them which results in damage to the body's own tissue. Depending upon the organ system involved, several autoimmune conditions have been identified. These defense reactions may be directed both against individual cell constituents and against entire cells or organs.

An autoimmune disease results from an inappropriate immune response directed against a self antigen (an autoantigen), which is a deviation from the normal state of self tolerance. Self-tolerance arises when the production of T cells and B cells capable of reacting against autoantigens has been prevented by events that occur in the development of the immune system during early life. The cell surface proteins that play a central role in regulation of immune responses through their ability to bind and present processed peptides to T cells are the major histocompatibility complex (MHC) molecules.

Allergies are known to be the result of hypersensitivity towards certain substances, the allergens, which gives rise to an over-reaction of the immune system. In other words, affected subjects react to certain substances (the allergens) with specific symptoms as a defense against the allergen.

### DISCUSSION OF RELATED ART

Attempts to treat autoimmune disorders caused by inappropriate reactions of the immune system with non-specifically acting immunosuppressants have proved entirely unsatisfactory as the use of immunosuppressants brings about a general inhibition of inflammatory reactions which may go as far as to shut down large parts of the immune system, so resulting in the occurrence of many side-effects, for example toxic damage, increased susceptibility to infectious diseases and increased risk of the occurrence of malignant diseases.

The alternative approach of avoiding the side-effects associated with the use of non-specifically acting immunosuppressants by using selective suppression (c.f. Ann. Neurol. 37 Suppl. 1, 87-101), with action being purposefully and specifically taken against the allergens or autoantigens at various points in the defense reaction, also resulted in less than satisfying success.

One of these methods is based upon the oral or inhalatory administration of autoantigens or allergens specific to the particular disorder. While it is indeed possible in this manner to reinduce or induce the body's tolerance to the autoantigens or allergens which have hitherto initiated an immune response, the overall success rate of this patient desensitisation is limited because the desensitisation is inadequate (Ann. N. Y. Acad. Sci. 778, 1-27; Ann. N. Y. Acad. Sci. 778, 243-250; Science 261, 1727-1730; Annu. Rev. Med. 48, 341-351).

The mechanism of oral reinduction or induction of tolerance by these substances is not yet completely understood. It may, however, be assumed that in the case of oral administration T cells (T lymphocytes) of the immune system, in particular the γδ-T cells as well as the bacterial flora of the gastrointestinal tract play a central role in establishing tolerance (The Journal of Immunology 158, 3610-3618; Res. Immunol. 147, 49-59; Immunology Letters 48, 97-102).

It was, however, entirely surprising that the reinduction or induction of tolerance achieved by oral or inhalatory administration of autoantigens or mixtures thereof or allergens specific to the disorder was greatly promoted if the autoantigens or allergens were administered in combination with bisphosphonic acids or the derivatives thereof. These combinations may thus successfully be used for the prevention and treatment of autoimmune disorders or allergies.

The use of bisphosphonic acids and of some of the derivatives thereof in pharmaceutical preparations is already known. The microbiostatic action of bisphosphonic acids (DE 3611522), the action thereof in the treatment of disorders of calcium and phosphate metabolism (DE 2534390, DE 2534391, DE 3334211, DE 3434667, DE 2745083), cytostatic action (DE 3425812), the lipid-reducing action thereof (Arzneimittelforschung 46, 759-762) and the ability thereof to stimulate immune cells (WO 97/38696 A1) are already known. The fact that bisphosphonic acids have an immunomodulatory action (WO 97/38696 A1) is furthermore known and has been used.

However, use of these compounds in monotherapeutically relevant concentrations is associated with numerous side-effects which are determined by the mode of administration. In the case of intravenous infusion, such side-effects are fever, flu-like symptoms with violent shivering, lymphopenia and thrombocytopenia and, in the case of oral administration, they are painful swallowing, oesophagitis, oesophageal erosion, oesophageal, ulceration, dyspepsia, diarrhoea etc. Moreover, oral treatment with bisphosphonates, for example, requires relatively large quantities of active substance and therapeutic success is still unsatisfactory (Drug-Saf. 14, 158-170).

It was thus not in the least obvious to use this group of compounds in combination with autoantigens or allergens in order to reinduce or induce the body's tolerance to autoantigens or allergens.

### DESCRIPTION OF THE INVENTION

The invention accordingly relates to a novel method of solving the hitherto unsolved problem of the prevention and treatment of autoimmune disorders and of allergies by means of pharmaceutical preparations, namely to use the autoantigens or allergens hitherto used to treat autoimmune disorders and allergies in combination with bisphosphonates or the derivatives thereof.

The invention relates to the use of bisphosphonic acids and the derivatives thereof for the production of pharmaceutical preparations for the prevention and treatment of autoimmune diseases or allergies, wherein the bisphosphonic acids and the derivatives thereof which are used are those of the general formula:

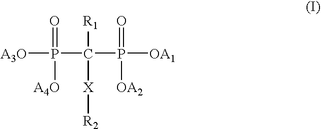

in which

A₁, A₂, A₃, and A₄ are independently selected from hydrogen, substituted or unsubstituted alkyl, substituted or unsubstituted aryl, substituted or unsubstituted aralkyl, substituted or unsubstituted cycloalkyl or a substituted or unsubstituted heterocyclic residue, a metal of main group 1, 2 or 3 of the periodic system, such as Na, K, Ca, Mg, Al, as well as substituted or unsubstituted ammonium or ammonium compounds derived from ethylenediamine or amino acids,

X, if present, may be selected from alkylene, alkenylene or hydroxyalkylene,

R₁ and R₂ are independently selected from hydrogen, —OH, —NH₂, a substituted or unsubstituted acyl, substituted or unsubstituted alkyl,substituted or unsubstituted aryl, substituted or unsubstituted cycloalkyl, substituted or unsubstituted aralkyl or a substituted or unsubstituted heterocyclic residue or —SR₃, Cl and —NR₃R₄, in which,

R₃ and R₄ are independently selected from hydrogen, OH, substituted or unsubstituted acyl, substituted or unsubstituted alkyl, substituted or unsubstituted aryl, substituted or unsubstituted aralkyl, substituted or unsubstituted cycloalkyl or a substituted or unsubstituted heterocyclic residue,

and the pharmaceutically compatible salts, esters thereof as well as salts of esters or compounds which, on administration, form the compounds to be administered as metabolites or catabolites, in combination with the specific autoantigens for the prevention and treatment of the particular autoimmune disorder, or in combination with the specific allergens for the prevention and treatment of the particular allergy, wherein, instead of the particular autoantigens or allergens, it is also possible to use fragments or derivatives thereof and the analogues or fragments thereof of the autoantigens or allergens, providing that these each exhibit the same immunological characteristics as the corresponding whole molecules, and wherein the bisphosphonic acids or the derivatives thereof and the autoantigens or allergens or fragments, derivatives or analogues thereof may be administered simultaneously or in succession.

The substances may here be administered both synchronously and with a delay by simultaneous or separate administration of the active substances.

From the group of bisphosphonic acids and the derivatives thereof of the general formula I, those bisphosphonic acids and the derivatives thereof which are preferred for use for the prevention and treatment of autoimmune disorders or allergies are of the general formula:

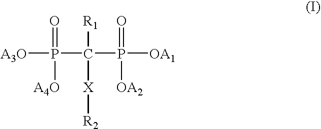

in which

A₁, A₂, A₃ and A₄ are independently selected from hydrogen, an alkyl residue having 1 to 12 carbon atoms, which may be branched or unbranched, an aryl residue, an aralkyl residue, cycloalkyl residue or a heterocyclic residue, wherein these residues may additionally be substituted by functional groups, a metal of main group 1, 2 or 3 of the periodic system, such as Na, K, Ca, Mg, Al, as well as substituted or unsubstituted ammonium or ammonium compounds derived from ethylenediamine or amino acids,

R₁ is selected from H, —OH, —NH₂,

X, if present, is selected from alkylene, alkenylene or hydroxyalkylene, in each case having 1 to 12 carbon atoms,

R₂ is selected from H, —OH, —NH₂, an alkyl residue having 1 to 12 carbon atoms, which may be branched or unbranched, an aryl residue, aralkyl residue, cycloalkyl residue or a heterocyclic residue, wherein these residues may additionally be substituted by functional groups, or —SR₃, Cl and —NR₃R₄, in which

R₃ and R₄ are independently selected from H, —OH, an alkyl residue having 1 to 12 carbon atoms, which may be branched or unbranched, an aryl residue, aralkyl residue, cycloalkyl residue or a heterocyclic residue, wherein these residues may additionally be substituted by functional groups.

Bisphosphonic acids and the derivatives thereof which have proved particularly effective are those of the general formula:

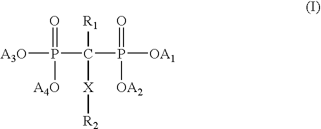

in which

A₁, A₂, A₃ and A₄ are independently selected from hydrogen, an alkyl residue having 1 to 12 carbon atoms, which may be branched or unbranched, an aryl residue, aralkyl residue, cycloalkyl residue or a heterocyclic residue, wherein these residues may additionally be substituted by functional groups, a metal of main group 1, 2 or 3 of the periodic system, such as Na, K, Ca, Mg, Al, as well as substituted or unsubstituted ammonium orammonium compounds derived from ethylenediamine or amino acids,

R₁ is selected from H and —OH,

X, if present, is selected from (CH₂)₁₋₅ and amidino,

R₂ is selected from a group consisting of:

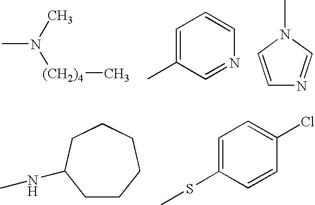

Some exampled of these include aminohydroxymethylidene bisphosphonic acid (AMP), 2-amino-1-hydroxyethylidene-1,1-bisphosphonic acid (AEP), 3-amino-1-hydroxypropylidene-1,1-bisphosphonic acid (pamidronic acid), 4-amino-1-hydroxybutylidene-1,1-bisphosphonic acid (alendronic acid), 6-amino-1-hydroxyhexylidene-1,1-bisphosphonic acid (AHP), amidinomethylenebisphosphonic acid (AIMP), 3-methylpentylamino-1-hydroxypropylidene-1,1-bisphosphonic acid (ibandronic acid), 2-(3-pyridinyl)-1-hydroxyethylidenebisphosphonic acid (risedronic acid), 1-hydroxy-2-(imidazol-1-yl)-ethylidene-1,1-bisphosphonic acid (zoledronic acid), cycloheptylaminomethylenediphosphonic acid (cimadronic acid),4-chlorophenylthiomethylene-1,1-bisphosphonic acid (tiludronic acid) and the derivatives thereof.

Autoimmune disorders and allergies are prevented and treated by combined use of a bisphosphonic acid or the derivatives thereof and an autoantigen which initiates the particular autoimmune disorder, such as for example in

multiple sclerosis with myelin-associated glycoprotein (MAG), myelin basic protein (MBP), myelin oligodendrocyte glycoprotein (MOG), myelin-oligodendrocytic basic protein (MOBP), oligodendrocyte-specific protein (OSP) and proteolipid protein (PLP), further preparations or extracts from nervous system tissue,

rheumatoid arthritis with type I, II or III collagen,

Hashimoto thyroiditis with thyroglobulin or fragments thereof,

myasthenia gravis with acetylcholine receptor protein or fragments thereof,

lupus erythematosus with DNA,

diabetes mellitus with extracts or preparations from islet cells, human insulin, or fragments of insulin peptide chains

primary biliary extracts or preparations from

cirrhosis liver tissue,

active chronic with liver cell extracts or

hepatitis preparations from liver tissue,

adrenalitis/Addison's with adrenal cortex extracts

disease or preparations from adrenal cortex tissue,

polymyositis extracts or preparations from skin tissue, extracts or preparations from muscle tissue,

dermatomyositis with extracts or preparations from muscle and/or skin tissue,

autoimmune haemolytic with haemopoetic cell line

anemia extracts,

myocarditis with extracts or preparations from heart muscle tissue or heart epithelium,

myopericarditis with extracts or preparations

scleroderma from skin tissue or cells

uveitis (phacouveitis) with preparations from eye lens proteins,

sympathetic ophthalmia) S-antigens, mixtures of S-antigen or fragments of S-antigen,

pemphigus vulgaris with extracts or preparations from skin tissue or cells,

pemphigoid with skin extracts or preparations from skin tissue or cells,

pernicious anemia with extracts or preparations from gastric cells, i.parietal cell extracts or preparations from parietal cells, intrinsic factor

autoimmune atrophic with gastric cell

gastritis extracts or preparations from gastric cells,

Crohn's disease with extracts or preparations from intestinal mucosa,

colitis ulcerosa with extracts or preparations from intestinal mucosa

or in allergies with extracts or preparations of the allergy-specific allergens.

Moreover, there are also known synthetic peptides in the state of the art, being suitable for the treatment of different autoimmune-disorder. Such synthetic peptides have the function of an autoantigen, being autoantigen-like—in accordance with the present invention.

Such suitable synthetic peptides are identified as follows:

Multiple sclerosis (MS): Copolymer 1 (Copaxone®)

Copolymer 1, also known as glatiramer acetate comprises the acetate salts of polypeptides containing L-glutamic acid, L-alanine, L-tyrosine and L-lysine. The average molar fraction of the amino acids are 0.141, 0.427, 0.095 and 0.338, respectively, and the average molecular weight of copolymer 1 is between 4,700 and 11,000 daltons. The efficacy against MS of copolymer-1 is disclosed in WO 98/30227.

However, the state of the art discloses further synthetic peptides having an autoantigen or autoantigen-like effect on the immune system. Recently, Strominger et al. identified several peptide compositions for the treatment of autoimmune diseases (US 2004/0006022A1, US 2004/0038887A1). Ben-Nun et al. (2005/0037422 A1) have provided further evidence that synthetic peptides display a therapeutic effect in the treatment of autoimmune-diseases, in particular MS.

Therefore, another object of the invention relates to synthetic peptides having an autoantigen or autoantigen-like function. Such peptides are incompletely listed as SEQ ID No. 1-145, referring to the teachings of Strominger et al. and Ben-Nun et al. (supra). In a further embodiment at least one synthetic peptide or a peptide of SEQ ID No. 1-145 being part of a fusion peptide having the function of an autoantigen, in particular wherein the autoantigen related to MS is selected from the group consisting of myelin-associated glycoprotein (MAG), myelin basic protein (MBP), myelin oligodendrocyte glycoprotein (MOG), myelin-oligodendrocytic basic protein (MOBP), oligodendrocyte-specific protein (OSP) and proteolipid protein (PLP).

In a further preferred embodiment one or more synthetic peptides in particular peptide selected of SEQ ID No. 1-145 serve as an epitope.

In a preferred embodiment the synthetic peptides may be part of a composition, consisting of a synthetic peptide according to the invention and a “natural/native” autoantigen.

Combined use is also taken to include cases in which allergens are already present or the body's own substances have become autoantigens. Such cases include, for example, Crohn's disease, in which components of the intestinal mucosa have become autoantigens as a result of the disease. In this case, in the event of oral or rectal administration, only the bisphosphonic acids or the derivatives thereof need to be administered. It is also unnecessary to administer the allergen if, during treatment, the affected subject is in an environment in which the allergy-specific allergen is already present (for example pollen during the pollen release season). In case of aerosolic pulmonary admission of the allergen the bisphosphonic acids or the derivatives thereof might be administered by inhalation.

Combined use may proceed not only by oral administration, for example by means of tablets etc., but also, for example, by rectal, inhalatory administration, by application onto the skin or mucous membranes. Preferred administration forms are oral and inhalatory administration and application onto the skin or mucous membranes.

Of these administration forms, inhalation has proved to be particularly gentle because elevated activity is achieved with only very small quantities of autoantigen or autoantigen-like peptide or allergen and bisphosphonic acid or the derivatives thereof and any possible side-effects of the active substances may accordingly be minimized.

The bisphosphonic acids and the derivatives thereof which are preferably used are those which are poorly resorbed, which include, for example, aminobisphosphonic acids and the derivatives thereof.

Preferred pharmaceutical compositions are tablets, sugar-coated pills, capsules, pills, granules, suppositories, solutions, suspensions and emulsions, pastes, ointments, gels, creams, lotions, powders and sprays. Tablets, sugar-coated pills, capsules, pills and granules may contain, apart from the active substances, conventional excipients, such as (a) fillers and extenders, for example starch, lactose, cane sugar, glucose, mannitol and silica, (b) binders, for example carboxymethylcellulose, alginates, gelatine, polyvinylpyrrolidone, (c) humectants, for example glycerol, (d) suspending agents, for example agar-agar, calcium carbonate and sodium carbonate, (e) dissolution retardants, for example paraffin and (f) resorption accelerators, for example quaternary ammonium compounds, (g) wetting agents, for example cetyl alcohol, glycerol monostearate, (h) adsorbents, for example kaolin and bentonite and (i) lubricants, for example talcum, calcium and magnesium stearate and solid polyethylene glycols or mixtures of the substances stated in (a) to (i).

The tablets, sugar-coated pills, capsules, pills and granules may be provided with conventional coatings and shells, which optionally contain opacifying agents, and may be of a composition such that they release the active substance, optionally with a delay, solely or preferentially in a specific part of the intestinal tract, wherein polymeric substances and waxes may, for example, be used as matrix materials.

The active substance or substances, optionally together with one or more of the above-stated excipients, may also assume microencapsulated form.

Apart from the active substance or substances, suppositories may contain conventional water-soluble or water-insoluble excipients, for example polyethylene glycols, fats, for example cocoa fat and higher esters (for example C₁₄ alcohol with C₁₆ fatty acid) or mixtures of these substances.

Apart from the active substance or substances, ointments, pastes, creams and gels may contain conventional excipients, for example animal and vegetable fats, waxes, paraffins, starch, tragacanth gum, cellulose derivatives, polyethylene glycols, silicones, bentonites, silica, talcum and zinc oxide or mixtures of these substances.

Apart from the active substance or substances, powders and sprays may contain conventional excipients, for example lactose, talcum, silica, aluminum hydroxide, calcium silicate and polyamide powder or mixtures of these substances. Sprays may additionally contain conventional propellants, for example chlorofluoro-carbons.

Apart from the active substance or substances, solutions and emulsions may contain conventional excipients, such as solvents, solubilizing agents and emulsifiers, for example water, ethyl alcohol, isopropyl alcohol, ethyl carbonate, ethyl acetate, benzyl alcohol, benzyl benzoate, propylene glycol, 1,3-butylene glycol, dimethylformamide, oils, in particular cottonseed oil, peanut oil, maize oil, olive oil, castor oil and sesame oil, glycerol, glycerol formal, tetrahydrofurfuryl alcohol, polyethylene glycols and sorbitan fatty acid esters or mixtures of these substances.

Apart from the active substance or substances, suspensions may contain conventional excipients, such as liquid diluents, for example water, ethyl alcohol, propylene glycol, suspending agents, for example ethoxylated isostearyl alcohols, polyoxyethylene sorbitol and sorbitan esters, microcrystalline cellulose, aluminium metahydroxide, bentonite, agar-agar and tragacanth gum or mixtures of these substances.

The stated formulation forms may also contain colorants, preservatives as well as with odor- and flavor-enhancing additives, for example peppermint oil and eucalyptus oil and sweeteners, for example saccharin.

Bisphosphonic acids or the derivatives thereof of the formula (I) are suitable for simultaneous, separate or temporally staged use with the autoantigens or allergens, and these compounds should accordingly be present in the pharmaceutical preparations listed above, preferably in a concentration of approx. 0.1 to 99.5 wt. %, relative to the complete mixture. The concentration of the autoantigens or allergens should be 0.1 to 99.5 wt. % in this case too.

Apart from the compounds of the formula (I) and the autoantigen or allergen, the pharmaceutical preparations listed above may also contain further pharmaceutical active substances.

The pharmaceutical preparations listed above are produced in the conventional manner using known methods, for example by mixing the active substance or substances with the excipient or excipients.

The stated preparations may be administered to humans or animals orally, rectally, intravaginally, topically (powders, ointments, drops) and in cavities and body cavities. Suitable preparations for oral treatment which may be considered are solutions and suspensions, gels, infusion formulations, emulsions, ointments or drops. Topical treatment may be performed using ophthalmological and dermatological formulations, silver and other salts, ear drops, eye ointments, powders or solutions. For animals, administration may be made by suitable formulation with feed or drinking water. Gels, pulverulent formulations, powders, tablets, delayed-release tablets, premixes, concentrates, granules, pellets, boli, capsules, aerosols, sprays, inhalatory preparations may also be used in humans and animals. The compounds according to the invention may furthermore be incorporated into other support materials, such as for example plastics (plastic chains for topical treatment), collagen or bone cement.

The quantities of the individual derivatives required to achieve the desired effect vary very widely. In general, it has proved advantageous in both human and veterinary medicine to administer the active substance or substances of the formula (I) in total quantities of approx. 0.5 to approx. 2000 mg per 24 hours, optionally in the form of two or more individual doses, in order to achieve the desired results. An individual dose preferably contains the active substance or substances in quantities of approx. 0.5 to approx. 2000 mg. It may, however, be necessary to deviate from the stated dosages, specifically as a function of the species and body weight of the subject to be treated, the nature and severity of the condition, the nature of the preparation and administration of the pharmaceutical preparation and the period of time or interval within which the preparation is administered.

It may accordingly be sufficient in some cases to use less than the above-stated quantity of active substance, while in other cases the active substance must be used in a quantity greater than that stated above. The person skilled in the art will establish the optimum dosage and mode of administration of the active substances in each case on the basis of his/her expertise.

When treating animals, the compounds to be used according to the invention may be given in the conventional concentrations and preparations together with feed or with feed preparations or with drinking water.

### EXAMPLES

Tablets are produced in a conventional manner well known to those skilled in the art using mixtures of

| | | | |
|---|---|---|---|
| 1. | 3-Amino-1-hydroxypropylidene-1,1- | 3 | mg |
| | bisphosphonate, disodium salt | | |
| | Bovine collagen, type II | 10 | mg |
| | Mannitol | 400 | mg |
| | Starch | 50 | mg |
| | Magnesium stearate | 10 | mg |
| 2. | 4-Amino-1-hydroxybutylidene-1,1- | 2.6 | mg |
| | bisphosphonate (monosodium salt),3H₂O | | |
| | Bovine collagen, type II | 10 | mg |
| | Mannitol | 400 | mg |
| | Starch | 50 | mg |
| | Magnesium stearate | 10 | mg |
| 3. | 3-Methylpentylamino-1-hydroxypropyl-idene- | | |
| | 1,1-bisphosphonate, monosodium salt, 1H₂O | 1.125 | mg |
| | Bovine collagen, type II | 10 | mg |
| | Mannitol | 400 | mg |
| | Starch | 50 | mg |
| | Magnesium stearate | 10 | mg |
| 4. | 3-Amino-1-hydroxypropylidene-1,1- | 1.5 | mg |
| | bisphosphonate, disodium salt | | |
| | Myelin basic protein(MBP) | 250 | mg |
| | Copolymer 1 | 25 | mg |
| | Mannitol | 400 | mg |
| | Starch | 50 | mg |
| | Magnesium stearate | 10 | mg |
| 5. | 4-Amino-1-hydroxybutylidene-1,1-bisphosphonate | 26 | mg |
| | (monosodium salt), 3H₂O | | |
| | Myelin basic protein (MBP) | 250 | mg |
| | Mannitol | 400 | mg |
| | Starch | 50 | mg |
| | Magnesium stearate | 10 | mg |
| 6. | 3-Methylpentylamino-1-hydroxypropylidene- | 1.125 | mg |
| | 1,1-bisphosphonate, monosodium salt, 1H₂O | | |
| | Myelin basic protein (MBP) | 250 | mg |
| | Mannitol | 400 | mg |
| | Starch | 50 | mg |
| | Magnesium stearate | 10 | mg |

Capsules are produced in a conventional manner well known to those
skilled in the art using mixtures of

| | | | |
|---|---|---|---|
| 7. | 3-Amino-1-hydroxypropylidene-1,1 - | 1.5 | mg |
| | bisphosphonate, disodium salt | | |
| | Myelin basic protein(MBP) | 250 | mg |
| | Proteolipid protein | 15 | mg |
| | Magnesium stearate | 15 | mg |
| 8. | 4-Amino-1-hydroxybutylidene-1,1- | 26 | mg |
| | Bisphosphonate (monosodium salt), 3H₂O | | |
| | Copolymer 1 | 150 | mg |
| | Proteolipid protein | 15 | mg |
| | Magnesium stearate | 15 | mg |
| 9. | 3-Methylpentylamino-1-hydroxypropylidene- | 1.125 | mg |
| | 1,1-bisphosphonate, monosodium salt, | | |
| | 1H₂O | | |
| | Myelin basic protein(MBP) | 250 | mg |
| | Proteolipid protein | 15 | mg |
| | Magnesium stearate | 15 | mg |
| 10. | 3-Amino-1-hydroxypropylidene-1,1- | 1.5 | mg |
| | bisphosphonate, disodium salt | | |
| | Bovine collagen, type II | 10 | mg |
| | Magnesium stearate | 15 | mg |
| 11. | 4-Amino-1-hydroxypropylidene-1,1- | 2.6 | mg |
| | bisphosphonate(monosodium salt), 3H₂O | | |
| | Bovine collagen, type II | 10 | mg |
| | Magnesium stearate | 15 | mg |
| 12. | 3-Methylpentylamino-1-hydroxypropyl- | 1.125 | mg |
| | idene-1,1-bisphosphonate, monosodium | | |
| | salt, 1H₂O | | |
| | Bovine collagen, type II | 10 | mg |
| | Magnesium stearate | 15 | mg |

wherein the above constituents are mixed together and then introduced
in conventional manner into a hard gelatine capsule.
A preparation for inhalation for a 2 ml dose is produced using:

| | | | |
|---|---|---|---|
| 13. | 4-Amino-1-hydroxybutylidene-1,1- | 1.3 | mg |
| | bisphosphonate (monosodium salt), 3H₂O | | |
| | Myelin basic protein | 15 | mg |
| | Copolymer 1 | 15 | mg |
| | β-Cyclodextrin hydrate | 7 | mg |
| | pH 7.2 phosphate buffer | 0.2 | ml |
| | water for injection; | | |
| 14. | 3-Amino-1-hydroxypropylidene-1,1- | 3 | mg |
| | bisphosphonate, disodium salt | | |
| | Myelin basic protein | 15 | mg |
| | 13-Cyclodextrin hydrate | 7 | mg |
| | pH 7.2 phosphate buffer | 0.2 | ml |
| | water for injection; | | |
| 15. | 3-Methylpentylamino-1-hydroxypropyl- | 0.25 | mg |
| | idene-1,1-bisphosphonate, monosodium salt, | | |
| | 1H₂O | | |
| | Copoloymer 1 | 15 | mg |
| | β-Cyclodextrin hydrate | 7 | mg |
| | pH 7.2 phosphate buffer | 0.2 | ml |
| | water for injection; | | |
| 16. | 4-Amino-1-hydroxybutylidene-1,1- | 1.3 | mg |
| | bisphosphonate (monosodium salt) | | |
| | 3H₂O Bovine collagen, type II | 10 | mg |
| | β-Cyclodextrin hydrate | 7 | mg |
| | pH 7.2 phosphate buffer | 0.2 | ml |
| | water for injection; | | |
| 17. | 3-Amino-1-hydroxyorioykudebeidene-1,1- | 30 | mg |
| | bisphosphonate, disodim salt | | |
| | Bovine collagen, type II | 10 | mg |
| | β-Cyclodextrin hydrate | 7 | mg |
| | pH 7.2 phosphate buffer | 0.2 | ml |
| | water for injection; | | |
| 18. | 3-Methylpentylamino-1-hydroxypropyl-idene- | 0.5 | mg |
| | 1,1-bisphosphonate, monosodium salt, | | |
| | 1H₂O | | |
| | Copolymer 1 | 10 | mg |
| | β-Cyclodextrin hydrate | 7 | mg |
| | pH 7.2 phosphate buffer | 0.2 | ml |
| | water for injection; | | |

The bisphosphonate (for example alendronate) and the autoantigen or autoantigen-like peptide or mixture of autoantigens or autoantigen-like peptides (for example MBP) are dissolved in a phosphate buffer solution and the beta-cyclodextrin hydrate is dissolved therein. The solution is made up to the desired volume with water for injection, sterilized by filtration and aseptically packaged in containers suitable for inhalation by atomization.

What is claimed is:

1. A medicament for treating an autoimmune disease, comprising a treatment enhancing amount of a first active ingredient when in combination with a second active ingredient, wherein the first active ingredient is selected from the group consisting of bisphosphonic acids corresponding to general formula (I) 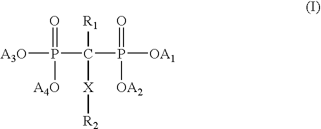
wherein
A1, A2, A3 and A4 are independently selected from the group consisting of hydrogen, substituted and unsubstituted alkyl, substituted and unsubstituted aryl, substituted and unsubstituted aralkyl, substituted and unsubstituted cycloalkyl, substituted and unsubstituted heterocyclic residues, metals of Groups I, II and III of the Periodic Table of the elements, and substituted and unsubstituted ammonium or ammonium compounds derived from ethylenediamine or amino acids,
X is absent or is selected from the group consisting of alkylene, alkenylene and hydroxyalkylene,
R1 and R2 are independently selected from the group consisting of H, OH, —NH₂, substituted and unsubstituted acyl, substituted and unsubstituted alkyl, substituted and unsubstituted aryl, substituted and unsubstituted cycloalkyl, substituted and unsubstituted aralkyl, substituted and unsubstituted heterocyclic residues, —SR₃, Cl and —NR₃R₄,
in which
R3 and R4 are independently selected from the group consisting of H, OH, substituted and unsubstituted acyl, substituted and unsubstituted alkyl, substituted and unsubstituted aryl, substituted and unsubstituted aralkyl, substituted and unsubstituted cycloalkyl and substituted and unsubstituted heterocyclic residues, their pharmaceutically compatible salts, esters thereof, salts of the esters and compounds, which upon administration from the compounds according to formula (I) or their salts or esters as metabolites or catabolites,
and a treatment enhancing amount of a second active ingredient when in combination with the first active ingredient, wherein said second active ingredient is at least one autoantigen specific for the autoimmune disease to be treated and selected from the group consisting of preparations or extracts from nervous system tissue, collagen, thyroglobulin or fragments thereof, acetylcholine receptor protein or fragments thereof, DNA, preparations or extracts from islet cells, human insulin or fragments of human insulin peptide chains, preparations or extracts from liver tissue, preparations or extracts from adrenal cortex tissue, preparations or extracts from skin tissue, preparations or extracts from muscle tissue, preparations or extracts from haemopoetic cell lines, preparations or extracts from heart tissue, preparations of eye lens proteins or parts thereof, S-antigens or parts thereof, preparations or extracts from gastric cells, preparations or extracts from parietal cells, intrinsic factor, and preparations or extracts from intestinal mucosa;
and/or autoantigen-like specific for the autoimmune disease represented by at least one synthetic peptide having the function of an autoantigen
and an excipient.

2. The medicament of claim 1, wherein the bisphosphonic acid is selected from the group consisting of
R₁ is selected from the group consisting of H, OH, —NH₂
R₂ is selected from the group consisting of H, OH, —NH₂, substituted and unsubstituted acyl, substituted and unsubstituted alkyl having 1 to 12 carbon atoms, substituted and unsubstituted aryl, substituted and unsubstituted cycloalkyl, substituted and unsubstituted aralkyl, substituted and unsubstituted heterocyclic residues, —SR₃, Cl and —NR₃R₄.

3. A medicament for treating an autoimmune disease, comprising
a treatment enhancing amount of a first active ingredient when in combination with a second active ingredient, wherein the first active ingredient is selected from the group consisting of bisphosphonic acids corresponding to general formula (I) 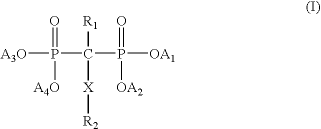
wherein
A1, A2, A3 and A4 are independently selected from the group consisting of hydrogen, substituted and unsubstituted alkyl, substituted and unsubstituted aryl, substituted and unsubstituted aralkyl, substituted and unsubstituted cycloalkyl, substituted
and unsubstituted heterocyclic residues, metals of Groups I, II and III of the Periodic Table of the elements, and substituted and unsubstituted ammonium or ammonium compounds derived from ethylenediamine or amino acids,
X is absent or is selected from the group consisting of (CH₂)₁₋₅ and amidino,
R₁ is selected from the group consisting of H and OH, and
R₂ is selected from the group consisting of —NH₂, 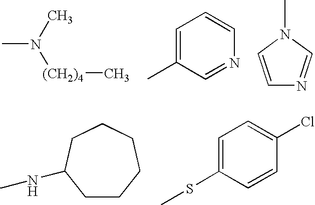
their pharmaceutically compatible salts, esters thereof, salts of the esters and compounds, which upon administration form the compounds according to formula (I) or their salts or esters as metabolites or catabolites, and
a treatment enhancing amount of a second active ingredient when in combination with the first active ingredient,
wherein said second active ingredient is at least one autoantigen specific for the autoimmune disease to be treated and selected from the group consisting of preparations or extracts from nervous system tissue, collagen, thyroglobulin or fragments thereof, acetylcholine receptor protein or fragments thereof, DNA, preparations or extracts from islet cells, human insulin or fragments of human insulin peptide chains, preparations or extracts from liver tissue, preparations or extracts from adrenal cortex tissue, preparations or extracts from skin tissue, preparations or extracts from muscle tissue, preparations or extracts from haemopoetic cell lines, preparations or extracts from heart tissue, preparations of eye lens proteins or parts thereof, S-antigens or parts thereof, preparations or extracts from gastric cells, preparations or extracts from parietal cells, intrinsic factor, and preparations or extracts from intestinal mucosa;
and/or autoantigen-like specific for the autoimmune disease represented by at least one synthetic peptide having the function of an autoantigen and
an excipient.

4. A medicament according to claim 1 wherein the medicament is present in a form selected from the group consisting of solid form, ointment, solution, and spray.

5. The medicament of claim 1, wherein the autoantigen is from a nervous system tissue extract and the autoantigen is myelin basic protein.

6. The medicament of claim 1, wherein the autoantigen is selected from the group consisting of collagen, thyroglobulin, acetylcholine receptor protein, human insulin, eye lens proteins, S-antigens, and intrinsic factor.

7. The medicament of claim 1, wherein the autoantigen is DNA.

8. The medicament of claim 1 wherein the bisphosphonic acid is an amino-1-hydroxyalkylidene-1,1-bisphosphonic acid wherein the alkyl is methyl, ethyl, propyl, butyl, or hexyl.

9. The medicament of claim 1 wherein the bisphosphonic acid is amidinomethylenebisphosphonic acid, risedronic acid, zoledronic acid, cimadronic acid, or tiludronic acid.

10. The medicament of claim 1 wherein the bisphosphonic acid is an amino-1-hydroxyalkylidene-1,1-bisphosphonic acid wherein the alkyl is methyl, ethyl, propyl, butyl, or hexyl.

11. The medicament of claim 1 wherein the bisphosphonic acid is an amino-1-hydroxyalkylidene-1,1-bisphosphonic acid wherein the alkyl is methyl, ethyl, propyl, butyl, or hexyl; and the autoantigen is from a nervous system tissue extract and is myelin basic protein.

12. The medicament of claim 1 wherein the bisphosphonic acid is an amino-1-hydroxyalkylidene-1,1-bisphosphonic acid wherein the alkyl is methyl, ethyl, propyl, butyl, or hexyl; and the autoantigen is collagen.

13. The medicament of claim 1 wherein the bisphosphonic acid is an amino-1-hydroxyalkylidene-1,1-bisphosphonic acid wherein the alkyl is methyl, ethyl, propyl, butyl, or hexyl; and the autoantigen is insulin.

14. The medicament of claim 1 wherein the synthetic peptide is a fusion peptide or a peptide composition being copolymer-1 or at least one peptide selected of the group SEQ ID No. 1-145.

15. The medicament of claim 1 wherein at least one synthetic peptide or at least one peptide selected of the group SEQ ID No. 1-145 being part of a fusion peptide having the function of an autoantigen.

16. The medicament of claim 1, wherein the autoantigen is related to multiple sclerosis and selected from the group consisting of myelin-associated glycoprotein (MAG), myelin basic protein (MBP), myelin oligodendrocyte glycoprotein (MOG), myelin-oligodendrocytic basic protein (MOBP), oligodendrocyte-specific protein (OSP) and proteolipid protein (PLP).

17. The medicament of claim 1 wherein the bisphosphonic acid and the autoantigen are present in a form for separate administration.
